SEQUENCE LISTING (6 sequences)

SEQ ID NO: 1 - DNA, 6198 bp, Artificial Sequence
  baculovirus L1X plasmid encoding for mutant HPV16/31L1 (pFastbac)
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcggatccatgagtctctggctcccctcggaggcaaccgtatacctccctcccgtcccagtgtctaaagtggtgtctaccgacgagtacgtcgcaagaactaacatctactaccatgccggcacttcacgtcttttggccgtgggacatccttactttccgattaagaagccaaacaacaataagattcttgtcccaaaagtttcgggtttgcaataccgcgttttccgcatccacctccgcgatccgaataagttcggcttcccagacacgtccttttacaatccggacactcaacgtttggtgtgggcctgtgtgggagtggaggtgggtcgtggacaaccgttgggcgttggaatttccggtcatcccctccttaacaagttggatgacaccgaaaatgcatcagcatacgctgcaaacgccggagtagataaccgcgagtgtatctctatggactataagcagacgcagctctgcctgattggttgtaagcctccaattggtgagcactggggcaaaggaagcccctgcaataacgtagccgtgaaccccggtgactgccctcctctggagctgataaacacggtcatccaagacggagatatggtcgataccggtttcggagctatggatttcactactctccaggctaacaagtccgaagtcccattggatatctgtacctcgatatgcaaataccccgattacatcaagatggttagcgaaccctacggcgactcactgttcttctatttgaggagagaacaaatgttcgtccgtcacctcttcaacagagctggtgcggtaggcgagaacgtccctacagacctctacatcaagggttctggtagcacagcgactctggcgaattcaaactatttccccactcccagtggaagcatggtgacctcagacgcccagatcttcaataagccctattggcttcagcgtgctcaaggccacaacaacggtatctgctggggcaatcaactgttcgtcacagttgtcgataccacgagatctaccaatatgtcgttgtgcgctgcgatttctacgtccgaacctacttacaagaacaccaacttcaaggagtacttgaggcatggtgaagaatacgatctgcaattcatcttccagctgtgcaagataacgctcaccgctgacgtaatgagctacatccactctatgaacagcactatcttggaggactggaactttggcctccagccgcctccaggcggaaccctggaggacacatatcgctttgttacctcccaggcgattgcttgccagaagcacacacctcctgctcccaaggaggaccctctcaagaaatacacattttgggaggtcaacttgaaagaaaagtttagtgccgatctggaccagtttcccttgggtaggaaattcctgctgcaggccggtctgaaggctaagccgaaattcacacttggcaagcgtaaagccactccaaccactagttccacctcaacaacagctaaacgtaagaagaggaaactttagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgg

SEQ ID NO: 2 - DNA, 6102 bp, Artificial Sequence
  baculovirus L2 plasmid encoding for HPV16L2 (pfastbac)
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcggatccatgcgccacaagaggtctgctaaacgtaccaaaagagcttctgcaactcagctctacaagacatgcaagcaggcaggcacgtgtcctcccgacatcattcccaaggtcgagggaaagaccattgctgatcaaatccttcagtacggatcgatgggcgtgttcttcggaggtctgggcattggtaccggttccggcacgggcggacgcaccggatacatccctcttggtactcgtcctcccacggccactgacacactggctcctgtccgtcctccgctcacagtggaccccgttggccctagtgacccctccatcgtcagccttgtggaagaaaccagctttatcgatgcgggagctcctactagcgttccatctatccctccggacgtgagcggtttctctatcactacttcaaccgatacaactcccgcgatcctcgatatcaacaacacggtcacgactgtcacaacccataacaatcctacttttaccgatccatcggtactgcaaccgcccacccctgctgaaaccggcggtcacttcacactgtcgtcatcaactatcagcactcataactacgaggagatcccgatggatacgttcatcgtgtcgaccaatcccaatactgttacctcctcaacccctatcccgggaagtaggcctgtagccaggttgggcctttacagtagaaccactcagcaggtcaaagtagttgaccctgcctttgttacaacacccaccaagttgattacctacgacaacccagcatacgagggcattgatgtcgataacacactctacttctcctctaacgacaatagcatcaatatcgctccagaccccgactttctggacatcgtcgccctgcaccgtcccgcactgacctcacgtaggaccggtatcagatattctcgcattggaaacaaacaaaccttgcgtactaggtctggcaagagcataggagcgaaggtacactattactatgatctctctacaatcgatccagctgaggagatcgaactccagacgattacgccgtccacatatactacgacttcccacgccgcatcacctacatccatcaacaacggcttgtacgacatctacgccgacgacttcatcactgatacttcgaccaccccagtgccatccgtgccatccacttctttgagtggttacatacccgccaataccactattcccttcggtggtgcctacaacattccactggtgtccggacccgacattcctatcaacatcacggaccaagccccttcacttattccaatagtacccggtagtccgcagtataccatcatagcggatgcgggcgacttctatctccatccaagttactacatgttgcgcaagcgccgcaagagactgccatacttcttctccgacgtgagcctggctgcttgatagaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgg

SEQ ID NO: 3 - DNA, 49 bp, Artificial Sequence
  shE7-1 Forward
caccaggaggatgaaatagatggttcgaaaaccatctatttcatcctcc

SEQ ID NO: 4 - DNA, 49 bp, Artificial Sequence
  shE7-1 Reverse
aaaaggaggatgaaatagatggttttcgaaccatctatttcatcctcct

SEQ ID NO: 5 - DNA, 10827 bp, Artificial Sequence
  Plasmid p16L1*L2 encoding 16/31 L1 (L1*) and L2 human codon optimized
ctagagccaccatgagcctgtggctgcccagcgaggccaccgtgtacctgccccccgtgcccgtgagcaaggtggtgagcaccgacgagtacgtggccaggaccaacatctactaccacgccggcaccagcaggctgctggccgtgggccacccctacttccccatcaagaagcccaacaacaacaagatcctggtgcccaaggtgagcggcctgcagtacagggtgttcaggatccacctgcccgaccccaacaagttcggcttccccgacaccagcttctacaaccccgacacccagaggctggtgtgggcctgcgtgggcgtggaggtgggcaggggccagcccctgggcgtgggcatcagcggccaccccctgctgaacaagctggacgacaccgagaacgccagcgcctacgccgccaacgccggcgtggacaacagggagtgcatcagcatggactacaagcagacccagctgtgcctgatcggctgcaagccccccatcggcgagcactggggcaagggcagcccctgcaccaacgtggccgtgaaccccggcgactgcccccccctggagctgatcaacaccgtgatccaggacggcgacatggtggacaccggcttcggcgccatggacttcaccaccctgcaggccaacaagagcgaggtgcccctggacatctgcaccagcatctgcaagtaccccgactacatcaagatggtgagcgagccctacggcgacagcctgttcttctacctgaggagggagcagatgttcgtgaggcacctgttcaacagggccggcgccgtgggcgagaacgtgcccaccgacctgtacatcaagggcagcggcagcaccgccaccctggccaacagcaactacttccccacccccagcggcagcatggtgaccagcgacgcccagatcttcaacaagccctactggctgcagagggcccagggccacaacaacggcatctgctggggcaaccagctgttcgtgaccgtggtggacaccaccaggagcaccaacatgagcctgtgcgccgccatcagcaccagcgagaccacctacaagaacaccaacttcaaggagtacctgaggcacggcgaggagtacgacctgcagttcatcttccagctgtgcaagatcaccctgaccgccgacgtgatgacctacatccacagcatgaacagcaccatcctggaggactggaacttcggcctgcagcccccccccggcggcaccctggaggacacctacaggttcgtgaccagccaggccatcgcctgccagaagcacaccccccccgcccccaaggaggaccccctgaagaagtacaccttctgggaggtgaacctgaaggagaagttcagcgccgacctggaccagttccccctgggcaggaagttcctgctgcaggccggcctgaaggccaagcccaagttcaccctgggcaagaggaaggccacccccaccaccagcagcaccagcaccaccgccaagaggaagaagaggaagctgtgaaagcttatcgataccgtcgacctcgacctgcagaagcttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattgcggccgctctagagccaccatgaggcacaagaggagcgccaagaggaccaagagggccagcgccacccagctgtacaagacctgcaagcaggccggcacctgcccccccgacatcatccccaaggtggagggcaagaccatcgccgaccagatcctgcagtacggcagcatgggcgtgttcttcggcggcctgggcatcggcaccggcagcggcaccggcggcaggaccggctacatccccctgggcaccaggccccccaccgccaccgacaccctggcccccgtgaggccccccctgaccgtggaccccgtgggccccagcgaccccagcatcgtgagcctggtggaggagaccagcttcatcgacgccggcgcccccaccagcgtgcccagcatcccccccgacgtgagcggcttcagcatcaccaccagcaccgacaccacccccgccatcctggacatcaacaacaccgtgaccaccgtgaccacccacaacaaccccaccttcaccgaccccagcgtgctgcagccccccacccccgccgagaccggcggccacttcaccctgagcagcagcaccatcagcacccacaactacgaggagatccccatggacaccttcatcgtgagcaccaaccccaacaccgtgaccagcagcacccccatccccggcagcaggcccgtggccaggctgggcctgtacagcaggaccacccagcaggtgaaggtggtggaccccgccttcgtgaccacccccaccaagctgatcacctacgacaaccccgcctacgagggcatcgacgtggacaacaccctgtacttcagcagcaacgacaacagcatcaacatcgcccccgaccccgacttcctggacatcgtggccctgcacaggcccgccctgaccagcaggaggaccggcatcaggtacagcaggatcggcaacaagcagaccctgaggaccaggagcggcaagagcatcggcgccaaggtgcactactactacgacctgagcaccatcgaccccgccgaggagatcgagctgcagaccatcacccccagcacctacaccaccaccagccacgccgccagccccaccagcatcaacaacggcctgtacgacatctacgccgacgacttcatcaccgacaccagcaccacccccgtgcccagcgtgcccagcaccagcctgagcggctacatccccgccaacaccaccatccccttcggtggcgcctacaacatccccctggtgagcggccccgacatccccatcaacatcaccgaccaggcccccagcctgatccccatcgtgcccggcagcccccagtacaccatcatcgccgacgccggcgacttctacctgcaccccagctactacatgctgaggaagaggaggaagaggctgccctacttcttcagcgacgtgagcctggccgcctgaaagctttttgaattctttggatccactagtggatcccccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcagaattctatcaaatatttaaagaaaaaaaaattgtatcaactttctacaatctctttcagaagacagaagcagagggaatacttcctaaatcattcaactaggccagcattaccttaataccggaactagaaaatgacattacaagaaaagaaaacaacagaccaatatctctcatgaacaaagatacaaacattttcaacaaaatattagcaaaaagaatccaagaatgtatcaaaaaatatacaccacaaccaagtagaatttattccagatatgtaagggtggttcaacgtttgaaaatcaattaacgtaatttgtcccatcaacaggttaaagaagaaaatcacatggtcatattgatagacacagaaaaagcatttgacaaaatttaacacccattcatgatgcaatctctcagtaaactaggaatagaggaaaacttcctcagcttgaatgtaccttcctctcaattttgctatgaacctgaaactcctcttaaaaaataaagtttttcatttaaaaagaaaacaaaaaacatggaggagcgttgatgtatctcattttagaccaatcagctatggatagttaggcgacagcacagatagctgctgtacttctgtttctggcaatgttccagactacatttaaaaaatttttaattatagacttgtacttaatgttcaagaaaaatatgaaaatggctttgccgtgttaatgctactcttttttaaaaaaaactaaagttcaaactttatttatatttcattagttttttagctactgttctttttctgttctgggatctcattcagaatgccacattacatataattctcatgtctccttgggttcctcttagttttgacagttcctcagacttttcttatttttgatgaccttgacagttttgaggagtactggttagatatagggtaatggtttttaaagtatatttgtcatgatttatactggggtaagggtttggggaggaagcccatggggtaaagtactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtcttttgggcttctatatacattttagaatgaggttggcaagttaacaaacagcttttttggggtgaacatattgactacaaatttatgtggaaagaaagtaccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaaggctagagtacttaatacgactcactatagg

SEQ ID NO: 6 - DNA, 10827 bp, Artificial Sequence
  p16sheLL plasmid sequence
ctagagccaccatgagcctgtggctgcccagcgaggccaccgtgtacctgccccccgtgcccgtgagcaaggtggtgagcaccgacgagtacgtggccaggaccaacatctactaccacgccggcaccagcaggctgctggccgtgggccacccctacttccccatcaagaagcccaacaacaacaagatcctggtgcccaaggtgagcggcctgcagtacagggtgttcaggatccacctgcccgaccccaacaagttcggcttccccgacaccagcttctacaaccccgacacccagaggctggtgtgggcctgcgtgggcgtggaggtgggcaggggccagcccctgggcgtgggcatcagcggccaccccctgctgaacaagctggacgacaccgagaacgccagcgcctacgccgccaacgccggcgtggacaacagggagtgcatcagcatggactacaagcagacccagctgtgcctgatcggctgcaagccccccatcggcgagcactggggcaagggcagcccctgcaccaacgtggccgtgaaccccggcgactgcccccccctggagctgatcaacaccgtgatccaggacggcgacatggtggacaccggcttcggcgccatggacttcaccaccctgcaggccaacaagagcgaggtgcccctggacatctgcaccagcatctgcaagtaccccgactacatcaagatggtgagcgagccctacggcgacagcctgttcttctacctgaggagggagcagatgttcgtgaggcacctgttcaacagggccggcgccgtgggcgagaacgtgcccgacgacctgtacatcaagggcagcggcagcaccgccaacctggccagcagcaactacttccccacccccagcggcagcatggtgaccagcgacgcccagatcttcaacaagccctactggctgcagagggcccagggccacaacaacggcatctgctggggcaaccagctgttcgtgaccgtggtggacaccaccaggagcaccaacatgagcctgtgcgccgccatcagcaccagcgagaccacctacaagaacaccaacttcaaggagtacctgaggcacggcgaggagtacgacctgcagttcatcttccagctgtgcaagatcaccctgaccgccgacgtgatgacctacatccacagcatgaacagcaccatcctggaggactggaacttcggcctgcagcccccccccggcggcaccctggaggacacctacaggttcgtgaccagccaggccatcgcctgccagaagcacaccccccccgcccccaaggaggaccccctgaagaagtacaccttctgggaggtgaacctgaaggagaagttcagcgccgacctggaccagttccccctgggcaggaagttcctgctgcaggccggcctgaaggccaagcccaagttcaccctgggcaagaggaaggccacccccaccaccagcagcaccagcaccaccgccaagaggaagaagaggaagctgtgaaagcttatcgataccgtcgacctcgacctgcagaagcttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattgcggccgctctagagccaccatgaggcacaagaggagcgccaagaggaccaagagggccagcgccacccagctgtacaagacctgcaagcaggccggcacctgcccccccgacatcatccccaaggtggagggcaagaccatcgccgaccagatcctgcagtacggcagcatgggcgtgttcttcggcggcctgggcatcggcaccggcagcggcaccggcggcaggaccggctacatccccctgggcaccaggccccccaccgccaccgacaccctggcccccgtgaggccccccctgaccgtggaccccgtgggccccagcgaccccagcatcgtgagcctggtggaggagaccagcttcatcgacgccggcgcccccaccagcgtgcccagcatcccccccgacgtgagcggcttcagcatcaccaccagcaccgacaccacccccgccatcctggacatcaacaacaccgtgaccaccgtgaccacccacaacaaccccaccttcaccgaccccagcgtgctgcagccccccacccccgccgagaccggcggccacttcaccctgagcagcagcaccatcagcacccacaactacgaggagatccccatggacaccttcatcgtgagcaccaaccccaacaccgtgaccagcagcacccccatccccggcagcaggcccgtggccaggctgggcctgtacagcaggaccacccagcaggtgaaggtggtggaccccgccttcgtgaccacccccaccaagctgatcacctacgacaaccccgcctacgagggcatcgacgtggacaacaccctgtacttcagcagcaacgacaacagcatcaacatcgcccccgaccccgacttcctggacatcgtggccctgcacaggcccgccctgaccagcaggaggaccggcatcaggtacagcaggatcggcaacaagcagaccctgaggaccaggagcggcaagagcatcggcgccaaggtgcactactactacgacctgagcaccatcgaccccgccgaggagatcgagctgcagaccatcacccccagcacctacaccaccaccagccacgccgccagccccaccagcatcaacaacggcctgtacgacatctacgccgacgacttcatcaccgacaccagcaccacccccgtgcccagcgtgcccagcaccagcctgagcggctacatccccgccaacaccaccatccccttcggtggcgcctacaacatccccctggtgagcggccccgacatccccatcaacatcaccgaccaggcccccagcctgatccccatcgtgcccggcagcccccagtacaccatcatcgccgacgccggcgacttctacctgcaccccagctactacatgctgaggaagaggaggaagaggctgccctacttcttcagcgacgtgagcctggccgcctgaaagctttttgaattctttggatccactagtggatcccccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcagaattctatcaaatatttaaagaaaaaaaaattgtatcaactttctacaatctctttcagaagacagaagcagagggaatacttcctaaatcattcaactaggccagcattaccttaataccggaactagaaaatgacattacaagaaaagaaaacaacagaccaatatctctcatgaacaaagatacaaacattttcaacaaaatattagcaaaaagaatccaagaatgtatcaaaaaatatacaccacaaccaagtagaatttattccagatatgtaagggtggttcaacgtttgaaaatcaattaacgtaatttgtcccatcaacaggttaaagaagaaaatcacatggtcatattgatagacacagaaaaagcatttgacaaaatttaacacccattcatgatgcaatctctcagtaaactaggaatagaggaaaacttcctcagcttgaatgtaccttcctctcaattttgctatgaacctgaaactcctcttaaaaaataaagtttttcatttaaaaagaaaacaaaaaacatggaggagcgttgatgtatctcattttagaccaatcagctatggatagttaggcgacagcacagatagctgctgtacttctgtttctggcaatgttccagactacatttaaaaaatttttaattatagacttgtacttaatgttcaagaaaaatatgaaaatggctttgccgtgttaatgctactcttttttaaaaaaaactaaagttcaaactttatttatatttcattagttttttagctactgttctttttctgttctgggatctcattcagaatgccacattacatataattctcatgtctccttgggttcctcttagttttgacagttcctcagacttttcttatttttgatgaccttgacagttttgaggagtactggttagatatagggtaatggtttttaaagtatatttgtcatgatttatactggggtaagggtttggggaggaagcccatggggtaaagtactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtcttttgggcttctatatacattttagaatgaggttggcaagttaacaaacagcttttttggggtgaacatattgactacaaatttatgtggaaagaaagtaccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggaaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaaggctagagtacttaatacgactcactatagg

(19) United States
(12) Patent Application Publication
(10) Pub. No.: US 2013/0012566 A1
(43) Pub. Date: Jan. 10, 2013

(54) Virion Derived Protein Nanoparticles For Delivering Diagnostic Or Therapeutic Agents For The Treatment of Alopecia

(75) Inventor: Elisabet De Los Pinos, Brookline, MA (US)

(73) Assignee: AURA BIOSCIENCES, INC., Cambridge, MA (US)

(21) Appl. No.: 13/251,742

(22) Filed: Oct. 3, 2011

Related U.S. Application Data
(60) Provisional application No. 61/506,140, filed on Jul. 10, 2011.

(51) Int. Cl. A61K 31/7088; A61P 17/14; A61P 17/00

(52) U.S. Cl. 514/44 A

(57) ABSTRACT

This invention relates to a transdermal delivery system for treating skin related diseases employing papilloma-derived protein nanoparticles to deliver drugs to the keratinocytes and basal membrane cells for the treatment of alopecia. The current invention presents an effective method for delivering small molecule nucleic acids to the epidermal cells.

### RELATED APPLICATIONS

This application claims the benefit of priority to U.S. Provisional Application No. 61/506,140 filed Jul. 10, 2011. The disclosure of the above applications are incorporated herein by reference.

### REFERENCE TO SEQUENCE LISTING

The Sequence Listing provides exemplary polynucleotide sequences of the invention. The traits associated with the used of the sequences are included in the Examples.

The Sequence Listing submitted as an initial paper is named AURA_{—}16_ST25.txt, is 45 kilobytes in size, and the Sequence Listing was created on 29 Nov. 2011. The copies of the Sequence Listing submitted via EFS-Web as the computer readable for are hereby incorporated by reference in their entirety.

### FIELD OF INVENTION

The invention relates to methods for loading protein nanoparticles with therapeutic, diagnostic or other agents, wherein the protein nanoparticles are based on viral proteins. More particularly, the present invention relates to a method for using papilloma-derived protein nanoparticles to deliver drugs to the keratinocytes and basal membrane cells for the treatment of alopecia or patterned hair loss.

### BACKGROUND OF THE INVENTION

Today there are a number of techniques available for the use of virus-like particles for the delivery of siRNA. A number of these techniques are discussed and outlined in a recent survey by Lund et al. entitled Pseudovirions as Vehicles for the Delivery of siRNA Pseudoviral Systems which is hereby incorporated by reference. As detailed by Lund, there are at present five distinct RNAi techniques being developed and tested for the delivery of siRNA.

The first three systems reviewed are the phagemid particles and Herpes Simplex Virus-1 (HSV) amplicons, in which the RNAi is derived from DNA, and the SV40 in vitro-packaged vectors. These three systems are examples of “trans-packaging systems in which viral machinery and proteins have been repurposed to package genetic material other than the original viral genome” (Lund et al. 401).

The fourth and fifth systems reviewed, the Influenza Virosome and the HVJ-E, a derivative of the Hemagglutinating Virus of Japan, are described as “viral envelope systems, in which native enveloped virus particles are inactivated and used to package other cargos” (Lund et al. 412).

Interestingly and most pertinent to the present invention, Lund details that the present methods for delivering siRNA know in the art are limited because, “Systems such as HSV amplicons and phagemid particles are only able to deliver DNA, which is inherently more risky than siRNA oliogomers, due to the possibility of integration into the host genome and insertional mutagenesis.”

Lund and other have raised additional concerns regarding the extent to which other available vectors such as the influenza virosome, HVJ-E, and SV40 in-vitro packaged pseudovirions, are inherently immunogenic.

With regards to other routes of administration, studies have attempted intradermal delivery of siRNA by injection. This method of delivery has shown effective knockdown of targeted gene expression but it is painful and the effects are localized to the injection site. Further, the delivery of siRNA through the stratum corneum is necessary but not sufficient for delivery to epidermal cells and additional steps must be taken to facilitate nucleic acid uptake by keratinocytes (and endosomal release) to allow access to the RNA-induced silencing complex. Because of these limitation, there is no existing method in the prior art which achieves both barrier disruption and intracellular delivery.

Accordingly, there is an unmet need for delivery strategies that increase drug half-life, bioavailability, selectivity and targeted, sustained release of key drugs. More specifically, there is an urgent need to deliver nucleic acid therapies to skin.

### SUMMARY OF INVENTION

The object of the present invention is to overcome the shortcomings disclosed in the prior art. The products of the current invention are pseudo-viruses derived from papillomaviruses, in particular from the genus betapapillomaviridae to deliver drugs to the keratinocytes and basal membrane cells for the treatment of skin related diseases. The present invention will allow for improved methods for administering treatment of skin diseases capable of targeting epithelial cells.

More specifically, the present invention describes the use of betaHPV pseudovirions and in particular type 5 betaHPV viral shells (L1 and L2) as an ideal delivery vehicle for the delivery of drugs to the skin for the treatment of alopecia.

Aspects of the invention relate to methods and compositions for producing papilloma-derived (e.g., human papilloma virus (HPV)-derived) protein nanoparticles containing one or more therapeutic or diagnostic agents. In some embodiments, aspects of the invention relate to methods and compositions for encapsulating an agent within a virus like particle (VLP) requiring an initial isolation of capsid proteins produced in a host cell system (e.g. yeast, mammalian cell, insect cell, E. coli) and subsequent reassembly in vitro.

The accompanying drawings, which are incorporated in and constitute part of the specification, illustrate various embodiments of the invention and together with the description, serve to explain the principles of the invention.

### BRIEF DESCRIPTION OF THE SEQUENCE LISTINGS AND DRAWINGS

FIG. 1A shows a table illustrating the dosing and adverse effects of Finasteride (Rogaine) and Minoxidil (Propecia) as currently manufactured.

FIG. 1B shows an illustrative example of the effects of DHT on the size of the hair follicle over time.

FIG. 1C shows an illustrative example of the effects of treating alopecia with commercially marketed form of Finasteride (Propecia).

FIG. 2 depicts shuttle vector information.

FIG. 3 depicts L1 capsid protein in various fractions from insect cell culture (T=total cell lysate, C=cytoplasmid fraction, TN=total nuclear fraction, SN=soluble nuclear fraction). Harvest times after baculovirus infection indicated.

FIG. 4 shims results from in vitro reassembly of capsid protein produced in insect cell culture. DLS demonstrates presence of capsid protein in form of monomers and oligomers after harvest from nuclear fraction (left) and appearance of well formed loaded VLPs after the reassembly procedure (right).

FIG. 5 is a graph showing the amount of luminescence/luciferase signal measured 48 hrs after treatment of HeLa cells with loaded VLP, where luminescence is reported on a scale of 0 to 30,000 units along the y-axis.

FIG. 6 is a graph the same data in FIG. 1A, showing the amount of luminescence/luciferase signal measured 48 hrs after treatment of HeLa cells with loaded VLP, where luminescence is reported on a scale of 0 to 20 units along the y-axis.

FIG. 7 shows a flow chart diagram of a preferred embodiment of the present invention.

(SEQ ID NO: 1) shows DNA sequence for baculovirus L1X plasmid encoding HPV16/31 L1 (pFastBac™).

(SEQ ID NO: 2) shows DNA sequence for baculovirus L2 plasmid encoding HPV16L2 (pFastBac™).

(SEQ ID NO: 3) shows forward primer DNA sequence used for generation of shE7-1 RNA construct.

(SEQ ID NO: 4) shows reverse primer DNA sequence used for generation of shE7-1 RNA construct.

(SEQ ID NO: 5) shows plasmid p16L1*L2 DNA sequence encoding 16/31 L1 (L1*) and L2 human codon-optimized.

(SEQ ID NO: 6) shows p16sheLL plasmid DNA sequence.

### DETAILED DESCRIPTION OF THE INVENTION

Alopecia means loss of hair from the head or body and includes baldness, a term generally reserved for pattern alopecia or androgenic alopecia (AGA). AGA is the most common cause of hair loss in humans, both male and female and its incidence is increasing 10% per decade. Researchers have determined that pattern hair loss is related to hormones called androgens, particularly an androgen called dihydrotestosterone (DHT). Men with androgenic alopecia typically have higher levels of 5-alpha-reductase, lower levels of total testosterone, higher levels of unbound/free testosterone, and higher levels of total free androgens including DHT (Denmark-Wahnefried 1997). 5-alpha-reductase is responsible for converting free testosterone into DHT. The genes for 5-alpha-reductase are known (Ellis 2005). The enzymes are present predominantly in the scalp and prostate. Levels of 5-alpha-reductase are one factor in determining levels of DHT in the scalp and drugs which interfere with 5-alpha-reductase (such as Finasteride, which inhibits the predominant type 2 isoform) have been approved by the FDA as treatments for hair loss.

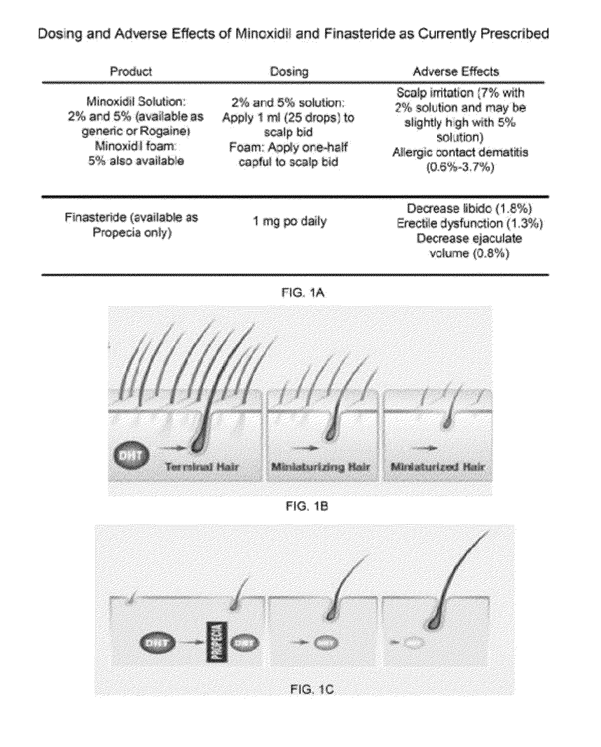

With reference now to FIG. 1A, a table illustrating the dosing and adverse effects of Finasteride (Rogaine manufactured by Pfizer) and Minoxidil (Propecia manufactured by Merck) as currently manufactured. As shown in FIG. 1A, the adverse side effects of the Minoxidil dosing include scalp irritation and allergic contact dermatitis. As further shown in FIG. 1A, the side effects associated with the DHT inhibitor, Finasteride, which when applied topically has very little penetration are more serious. The compounds and formulas of U.S. Pat. No. 6,268,377 which is hereby incorporated by reference, for the treatment of hyperandrogenic condition, include the 5-alpha-reductase inhibitors and calcium channel blocking agents which may be combined in a single oral, topical, systemic or parenteral dosage formulation. As shown in FIG. 1A, the composition has a number of side effects that include: erectile dysfunction (ED) or impotence in up to 18.5 percent of people, decreased libido up to 10 percent, abnormal ejaculation, including decreased ejaculate amount, up to 7.2 percent, and breast enlargement up to 2.2 percent.

With reference now to FIG. 1B, an illustrative example of the effects of DHT on the size of the hair follicle over time will be discussed. As shown in FIG. B, DHT may contribute to the shortening of the growth phase of the hair follicle causing them to shrink until there are fewer visible hairs left in the scalp.

With reference now to FIG. 1C, an illustrative example of the effects of treating Alopecia with commercially marketed Finasteride or Propecia will be discussed. As shown in FIG. 1C, Propecia blocks the formation of DHT which is intended to inhibit the development of male pattern baldness. However, the ineffectiveness of Propecia in treating male patterned baldness and the adverse effects associated with the currently prescribed method of dosing and treating male pattern baldness indicate that a more effective treatment is needed.

Assembly of Particles

To assemble the biological, pharmaceutical or diagnostic components to a described biological cargo-laden nanoparticles used as a carrier, the components can be associated with the nanoparticles through a linkage. By “used as a carrier associated with”, it is meant that the component is carried by the nanoparticles. The component can be dissolved and incorporated in the nanoparticles non-covalently. Preferred and illustrative methods for creating, loading and assembling particles for use with the present are taught in following applications which are hereby incorporated by reference in their entirety: PCT/US2009/00429 entitled “HVP PARTICLES AND USES THEREOF;” PCT/IB2010/002654, Nov. 24, 2010 entitled “TARGETING OF PAPILLOMA VIRUS GENE DELIVERY PARTICLES;” U.S. Provisional Application No. 61/417,031 entitled “METHOD FOR LOADING HPV PARTICLES;” and U.S. Provisional Application No. 61/491,774 entitled “PAPILLOMA-DERIVED PROTEIN NANOSPHERES FOR DELIVERING DIAGNOSTIC OR THERAPEUTIC AGENTS.”

In some embodiments, aspects of the invention relate to methods and compositions for producing protein nanoparticles that contain therapeutic and/or diagnostic agents for delivery to a subject. Methods and compositions have been developed for effectively encapsulating therapeutic and/or diagnostic agents within papilloma virus proteins (e.g., HPV proteins) that can be used for delivery to a subject (e.g., a human subject).

In some embodiments, it has been discovered that it is useful to isolate L1 and L2 capsid proteins directly from host cells as opposed to disassembling VLPs that were isolated from host cells. L1 and L2 capsid proteins that are isolated directly from cells can be used in in vitro assembly reactions to encapsulate a therapeutic or diagnostic agent. This avoids the additional steps of isolating and disassembling VLPs. This also results in a cleaner preparation of L1 and L2 proteins, because there is a lower risk of contamination with host cell material (e.g., nucleic acid, antigens or other material) that can be contained in VLPs that are isolated from cells.

In some embodiments, it has been discovered that expressing L1 and/or L2 proteins intracellularly in the presence of a therapeutic or diagnostic agent can be useful in the production of a loaded VLP intracellularly that encapsulates the agent.

In some embodiments, it is useful to independently produce L1 and L2 capsid proteins. In some embodiments, they can be produced from two independent nucleic acids (e.g., different vectors). In some embodiments, they can be produced in the same cell (e.g., using two different vectors within the same cell). In some embodiments, they can be produced in different cells (e.g., different host cells of the same type or different types of host cell). This approach allows the ratio of L1 and L2 proteins to be varied for either in vitro or intracellular assembly. This allows VLPs to be assembled (e.g., in vitro or intracellularly) with higher or lower L1 to L2 ratios than in a wild type VLP. This may have benefits in the use of HPV nanoparticles as delivery vehicles for therapeutic agents. A higher ratio of L2 in the assembled structure may allow the resultant VLP to have a higher nucleic acid binding affinity and a better efficiency in delivering these intracellularly.

Capsid Proteins:

In some embodiments, L1 and 1.2 proteins are expressed in a host cell system (e.g., both in the same host cell or independently in different host cells). L1 and/or L2 are isolated from nuclei of the host cells. In some embodiments, certain L1 and/or L2 structures that are formed during cellular growth (e.g., during the fermentation process) are disrupted. Any suitable method may be used. In some embodiments, sonication may be used (e.g., nuclei may be isolated and then sonicated). Capsid proteins then may be purified using any suitable process. For example, in some embodiments, capsid proteins may be purified using chromatography.

Isolated capsid proteins can then be used as described herein in a cell free system to assemble together with different payloads to create superstructures that contain a drug or diagnostic agent in its interior.

It should be appreciated that directly isolating capsid proteins (as opposed to isolating and disassembling VLPS) provides several benefits. In some embodiments, there is a reduced risk of encapsulating and transferring genetic information (DNA, RNA) from the host cell to the treated subject. In certain embodiments, de-novo assembly of VLPs during the assembly procedure ensures formation of a larger percentage of loaded VLPs as opposed to using already-formed VLPs for loading where a certain fraction can remain unloaded.

Cellular Production:

In some embodiments, one or more therapeutic or diagnostic agents may be loaded intracellularly by expressing L1 and/or L2 in the presence of intracellular levels of one or more agents of interest.

In some embodiments, this method is used for encapsulating a silencing plasmid which will encode for expression of short hairpin RNA (shRNA). In some embodiments, this plasmid will have a size of 2 kB-6 kB. However, any suitable size may be used. In some embodiments, a plasmid is designed to be functional within the cells of the patient or subject to be treated (to which the loaded VLP is administered). Accordingly, the plasmid will be active within the target cells resulting in knockdown of the targeted gene(s).

In some embodiments, this method may be used to encapsulate short interfering RNA (siRNA) or antisense nucleic acids (DNA or RNA) transfected into the host cells (e.g., 293 cells or other mammalian or insect host cells) during the production of the VLPs.

Accordingly, loaded VLPs may be produced intracellularly to provide gene silencing functions when delivered to a subject.

It should be appreciated that there are several benefits to this method. In some embodiments, encapsulation of RNA interference (RNAi) constructs into VLPs allows for very efficient transfer of RNAi or Antisense nucleic acid into target cells.

Independent Expression Vectors:

In some embodiments, L1 and L2 proteins are expressed in a host cell system (e.g. mammalian cells or insect cells) from independent expression nucleic acids (e.g., vectors, for example, plasmids) as opposed to both being expressed from the same nucleic acid.

It should be appreciated that the expression of L1 and L2 from independent plasmids allows the relative levels of L1/L2 VLP production to be optimized for different applications and to obtain molecular structures with optimal delivery properties for different payloads. In some embodiments, a variety of VLP structures can be produced to tit the needs of the different classes of payloads (e.g., DNA, RNA, small molecule, large molecule) both in terms of charge and other functions (e.g. DNA binding domains, VLP inner volume, endosomal release function). VLPs with a higher content of L2 protein will be better to bind nucleic acids (L2 contains a DNA binding domain) whereas VLPs with a smaller content of L2 protein will be better for other small molecules. VLPs with different ratios of L1:L2 protein will have different inner volumes that will allow a higher concentration of drug to be encapsulated. In some embodiments, the release of payload into the cell will also be modulated. In some embodiments, structures containing more L2 protein may have a higher ability to transfer nucleic acids intracellularly. It should be appreciated that different ratios of L1/1.2 may be used. In some embodiments, ratios may be 1:1, 1:2, 1:4, 1:5, 1:20 or 1:100. However, other ratios may be used as aspects of the invention are not limited in this respect.

In some embodiments, each separate expression nucleic acid encodes an L1 (but not an L2) or an L2 (but not an L1) sequence operably linked to a promoter. In some embodiments, other suitable regulatory sequences also may be present. The separate expression nucleic acids may use the same or different promoters and/or other regulatory sequences and/or replication origins, and/or selectable markers. In some embodiments, the separate nucleic acids may be vectors (e.g., plasmids, or other independently replicating nucleic acids). In some embodiments, separate nucleic acids may be independently integrated into the genome of a host cell (e.g., a first nucleic acid integrated and a second nucleic acid on a vector, two different nucleic acids integrated at different positions, etc.). In some embodiments, the relative expression levels of L1 and L2 may be different in different cells, different using different expression sequences, independently regulated, or a combination thereof.

Variant HPV Proteins Having Reduced Immunogenicity:

In some embodiments, an expression vector is used to produce a mutant L1 or L2 protein. In some embodiments, a mutant HPV16L1 protein (called L1*) is expressed along with L2 in a host system (e.g., a 293 cell system). These can then be isolated and assembled as described herein to encapsulate a therapeutic or diagnostic payload (e.g. therapeutic plasmid, siRNA, small molecule drugs, etc.).

In some embodiments, loaded VLPs are produced using certain L1 and/or L2 variant sequences that are not recognized by existing antibodies against HPV (e.g. HPV16L1) that might be present in patients who have an ongoing HPV infection or who have received the vaccine. It also should be appreciated that loaded VLPs can be produced using L1 and/or L2 proteins that are modified to reduce antigenicity against other HPV serotype antibodies and/or to target the loaded VLP to particular organs or tissues (e.g., lung) or cells or subcellular locations.

Accordingly, certain aspects of the invention relate to methods for loading VLPs with therapeutic, diagnostic or other agents. In certain embodiments, the papilloma virus particles are HPV-VLP. In certain embodiments, the methods described herein utilize HPV-VLP that contain one or more naturally occurring HPV capsid proteins (e.g., L1 and/or L2 capsid proteins). HPV-VLP may be comprised of capsid protein oligomers or monomers.

A “VLP” refers to the capsid-like structures which result upon assembly of a HPV L1 capsid protein alone or in combination with a HPV L2 capsid protein. VLPs are morphologically and antigenically similar to authentic virions. VLPs lack viral genetic material (e.g., viral nuclei acid), rendering the VLP non-infectious. VLP may be produced in vivo, in suitable host cells, e.g., mammalian, yeast, bacterial and insect host cells.

A “capsomere” refers to an oligomeric configuration of L1 capsid protein. Capsomeres may comprise at least one L1 (e.g., a pentamer of L1).

A “capsid protein” refers to L1 or L2 proteins that are involved in building the viral capsid structure. Capsid proteins can form oligomeric structures i.e. pentamers, trimers or be in single units as monomers.

In some embodiments, a VLP can be loaded with one or more medical, diagnostic and/or therapeutic agents, or a combination of two or more thereof. In some embodiments, the methods described herein utilize HPV-VLP that contain one or more variant capsid proteins (e.g., variant L1 and/or L2 capsid proteins) that have reduced or modified immunogenicity in a subject. Examples of variant capsid proteins are described in WO 2010/120266. The modification may be an amino acid sequence change that reduces or avoids neutralization by the immune system of the subject. In some embodiments, a modified HPV-VLP contains a recombinant HPV protein (e.g., a recombinant L1 and/or L2 protein) that includes one or more amino acid changes that alter the immunogenicity of the protein in a subject (e.g., in a human subject). In some embodiments, a modified HPV-VLP has an altered immunogenicity but retains the ability to package and deliver molecules to a subject.

In certain embodiments, amino acids of the viral wild-type capsid proteins, such as L1 and/or L1+L2, assembling into the HPV-VLP, are mutated and/or substituted and/or deleted. In certain embodiments, these amino acids are modified to enhance the positive charge of the VLP interior. In certain embodiments, modifications are introduced to allow a stronger electrostatic interaction of nucleic acid molecules with one or more of the amino acids facing the interior of the VLP and/or to avoid leakage of nucleic acid molecules out of the VLP. Examples of modifications are described in WO2010/120266. It should be appreciated that any modified HPV-VLP may be loaded with one or more agents. Such particles may be delivered to a subject without inducing an immune response that would be induced by a naturally-occurring HPV.

In certain embodiments, HPV-VLP, loaded according to the methods described herein, are useful for delivering one or more therapeutic agents to diseased tissue (e.g., diseased mucosal tissue). In some embodiments, a diseased tissue (e.g., mucosal tissue, epithelial tissue, or endothelial tissue) may be an infected tissue (e.g., infected with a virus such as HPV or HSV). In some embodiments, the mucosal tissue is cervical tissue and the disease is dysplasia or cancer (e.g., cervical dysplasia, cervical cancer, for example associated with persistent HPV infection). In some embodiments, HPV-VLP, loaded according to the methods described herein, may be used to deliver compositions to other tissues (e.g., epidermis). In some embodiments, HPV-VLP may be used to treat HPV related diseases. In some embodiments, HPV-VLP, loaded according to the methods described herein, may be used to deliver therapeutic agents to treat a disease or condition, and/or may be used to deliver diagnostic agents to diagnose other diseases or conditions. For example, quantum dots, metals, and/or other imaging agents may be loaded into VLP according to the methods described herein and delivered. In some embodiments, agents may be used to track early stage diseases (e.g., early stage metastasis). It should be appreciated that any suitable therapeutic, diagnostic and/or other medical agent may be loaded into VLP according to the methods described herein and delivered to a subject. Examples of administration of VLP to subjects are described for example in U.S. Pat. No. 7,205,126, incorporated herein by reference.

In some embodiments, HPV-VLPs that are modified to display epitopes from two or more different naturally-occurring HPV variants are used in the agent loading methods described herein. Such modified VLPs may be used to provide immunization against infection by any of two or more naturally occurring HPV variants.

In some embodiments, HPV-VLPs comprise viral L1 capsid proteins. In some embodiments, HPV-VLPs comprise viral L1 capsid proteins and viral L2 capsid proteins. The L1 and/or L2 proteins may, in some embodiments, be wild-type viral proteins. In some embodiments, L1 and/or L2 capsid proteins may be altered by mutation and/or deletion and/or insertion so that the resulting L1 and/or L2 proteins comprise only ‘minimal’ domains essential for assembly of a VLP. In some embodiments, L1 and/or L2 proteins may also be fused to other proteins and/or peptides that provide additional functionality. Examples of modifications are described for example in U.S. Pat. No. 6,991,795, incorporated herein by reference. These other proteins may be viral or non-viral and could, in some embodiments, be for example host-specific or cell type specific. It should be appreciated that VLPs may be based on particles containing one or more recombinant proteins or fragments thereof (e.g., one or more HPV membrane and/or surface proteins or fragments thereof). In some embodiments, VLPs may be based on naturally-occurring particles that are processed to incorporate one or more agents as described herein, as aspects of the invention are not limited in this respect. In certain embodiments, particles comprising one or more targeting peptides may be used. Other combinations of HPV proteins (e.g. capsid proteins) or peptides may be used as aspects of the invention are not limited in this respect.

In some embodiments, viral wild-type capsid proteins are altered by mutations, insertions and deletions. All conformation-dependent type-specific epitopes identified to date are found on the HPV-VLP surface within hyper-variable loops where the amino acid sequence is highly divergent between HPV types, which are designated BC, DE, EF, FG and HI loops. Most neutralizing antibodies are generated against epitopes in these variable loops and are type-specific, with limited cross-reactivity, cross-neutralization and cross-protection. Different HPV serotypes induce antibodies directed to different type-specific epitopes and/or to different loops. Examples of variant capsid proteins are described in WO 2010/120266.

In certain embodiments, viral capsid proteins, HPV L1 and/or L2, are mutated at one or more amino acid positions located in one or more hyper-variable and/or surface-exposed loops. The mutations are made at amino acid positions within the loops that are not conserved between HPV serotypes. These positions can be completely non-conserved, that is that any amino acid can be at this position, or the position can be conserved in that only conservative amino acid changes can be made.

In certain embodiments, L1 protein and L1+L2 protein may be produced recombinantly. In certain embodiments, recombinantly produced L1 protein and L1+L2 protein may self-assemble to form virus-like particles (VLP). Recombinant production may occur in a bacterial, insect, yeast or mammalian host system. L1 protein may be expressed or L1+L2 protein may be co-expressed in the host system.

Cellular hosts that are useful for expressing and purifying HPV L1 and/or L2 recombinant viral capsid proteins are known in the art. For example, HPV L1 and/or L2 proteins may be expressed in Spodoptera frugiperla (Sf21) cells. Baculoviruses encoding the L1 and/or L2 gene of any HPV or recombinant versions thereof from different serotypes (e.g., HPV16, HPV18, HPV31, and HPV58) may be generated as described in Touze et al., FEMS Microbiol. Lett. 2000; 189:121-7; Touze et al., J. Clin. Microbiol. 1998; 36:2046-51); and Combita et al., FEMS Microbiol. Lett. 2001; 204(1): 183-8. HPV L1 and/or L2 genes may be cloned into a plasmid, such as pFastBac1 (Invitrogen). Sf21 cells may be maintained in Grace's insect medium (Invitrogen) supplemented with 10% fetal calf serum (FCS, Invitrogen) and infected with recombinant baculoviruses and incubated at 27° C. Three days post infection, cells can be harvested and VLP can be purified. For example, cells may be resuspended in PBS containing Nonidet P40 (0.5%), pepstatin A, and leupeptin (1 μg/ml each, Sigma Aldrich), and allowed to stand for 30 min at 4° C. Nuclear lysates may then be centrifuged and pellets can be resuspended in ice cold PBS containing pepstatin A and leupeptin and then sonicated. Samples may then be loaded on a CsCl gradient and centrifuged to equilibrium (e.g., 22 h, 27,000 rpm in a SW28 rotor, 4° C.). CsCl gradient fractions may be investigated for density byrefractometry and for the presence of L1/L2 protein by electrophoresis in 10% sodium dodecyl sulfate-polyacrylamide gel (SDS-PAGE) and Coomassie blue staining. Positive fractions can be pooled, diluted in PBS and pelleted e.g., in a Beckman SW 28 rotor (3 h, 28,000 rpm, 4° C.). After centrifugation, VLP can be resuspended in 0.15 mol/L NaCl and sonicated, e.g. by one 5 second burst at 60% maximum power. Total protein content may be determined.

Viral capsid proteins may also be expressed using galactose-inducible Saccharomyces cerevisiae expression system. Leucine-free selective culture medium used for the propagation of yeast cultures, yeast can be induced with medium containing glucose and galactose. Cells can be harvested using filtration. After resuspension, cells may be treated with Benzonase and subsequently mechanically disrupted (e.g., using a homogenizer). Cell lysate may be clarified using filtration. An exemplary protocol can be found in Cook et al. Protein Expression and Purification 17, 477-484 (1999).

Buck et al. Virol. 78, 751-757, 2004) reported the production of papilloma virus-like particles (VLP) and cell differentiation-independent encapsidation of genes into bovine papillomavirus (BPV) L1 and L2 capsid proteins expressed in transiently transfected mammalian cells, 293TT human embryonic kidney cells, which stably express SV40 large T antigen to enhance replication of SV40 origin-containing plasmids. Pyeon et al. reported a transient transfection method that achieved the successful and efficient packaging of full-length HPV genomes into HPV16 capsids to generate virus particles (PNAS 102, 9311-9316 (2005)). Transiently transfected cells (e.g., 293 cells, for example 293T or 293TT cells) can be lysed by adding Brij58 or similar nonionic polyoxyethylene surfactant detergent, followed by benzonase and exonuclease V and incubating at 37° C. for 24 h to remove unpackaged cellular and viral DNA and to allow capsid maturation. The lysate can be incubated on ice with 5 M NaCl and cleared by centrifugation. VLP can be collected by high-speed centrifugation.

Capsid proteins may also be expressed in E. coli. In E. coli, one important potential contaminant of protein solutions is endotoxin, a lipopolysaccharide (LPS) that is a major component of the outer membrane of Gram-negative bacteria (Schädlich et al. Vaccine 27, 1511-1522 (2009)). For example, transformed BL21 bacteria may be grown in LB medium containing 1 mM ampicillin and incubated with shaking at 200 rpm at 37° C. At an optical density (OD₆₀₀ nm) of 0.3-0.5, bacteria can be cooled down and IPTG may be added to induce protein expression. After 16-18 h bacteria may be harvested by centrifugation. Bacteria may be lysed by homogenizing, lysates may be cleared, capsid proteins purified and LPS contamination removed, using e.g., chromatographic methods, such as affinity chromatography and size exclusion chromatography. LPS contamination may also be removed using e.g., 1% Triton X-114.

In certain embodiments, VLPs are loaded with the one or more therapeutic agents. After isolation of L1 and L2 capsid proteins which may be in the form of monomers or oligomers, VLPs may be assembled and loaded by disassembling and reassembling L1 or L1 and L2 viral capsid proteins, as described herein. Salts that are useful in aiding disassembly/reassembly of viral capsid proteins into VLPs, include Zn, Cu and Ni, Ru and Fe salts. In some embodiments, VLPs may be loaded with one or more therapeutic agents.

Loading of VLPs with agents utilizing a disassembly-reassembly method has been described previously, for example in U.S. Pat. Nos. 6,416,945 and WO 2010/120266, incorporated herein by reference. Generally, these methods involve incubation of the VLP in a buffer comprising EGTA and DTT. Under these conditions, VLP completely disaggregated into structures resembling capsid proteins in monomeric or oligomeric form. A therapeutic or diagnostic agent, as described herein, may then be added and the preparation diluted in a buffer containing DMSO and CaCl₂ with or without ZnCl₂ in order to reassemble the VLP. The presence of ZnCl₂ increases the reassembly of capsid proteins into VLP. In some embodiments, one or more of these reassembly methods may be used to assemble capsid proteins to form VLPs that encapsulate one or more agents without requiring an initial VLP disassembly procedure, as described herein.

In certain embodiments, VLP are loaded with the one or more therapeutic agents. After isolation of L1 and L2 capsid proteins, these may mixed directly after purification from the host cell with the therapeutic agent and reassembled into loaded VLPs as described herein, the preparation diluted in a buffer containing DMSO and CaCl₂ with or without ZnCl₂ in order to reassemble the VLP. The presence of ZnCl₂ increases the reassembly of capsid proteins into VLP.

Surprisingly, it was found that certain ratios of a) Capsid protein to reaction volume,

b) agent to capsid protein, and/or c) agent to reaction volume lead to agent-loaded VLP (VLP comprising entrapped agent) that exhibit superior delivery of agent to target cells when compared to agent-loaded VLP prepared using previously described methods. VLP loaded with agents using the methods described herein, in certain embodiments, are able to deliver agent to 65%, 75%, 85%, 95%, 96%, 97%, 98%, or 99% of target cells. One non-limiting example of the improved method is exemplified in the Examples.

For example, VLP may be loaded with a nucleic acid using a method comprising: a) contacting a preparation of capsid proteins with the nucleic acid in a reaction volume, wherein i) the ratio of capsid protein to reaction volume ranges from 0.1 μg capsid protein per 1 μl reaction volume to 1 μg capsid protein per 1 μl reaction volume; ii) the ratio of nucleic acid to capsid protein ranges from 0.1 μg nucleic acid per 1 μg capsid protein to 10 μg nucleic acid per 1 μg capsid protein; and/or iii) the ratio of nucleic acid to reaction volume ranges from 0.01 μg nucleic acid per 1 μl reaction volume to 10 μg nucleic acid per 1 μl reaction volume, and b) reassembling the capsid proteins to form a VLP, thereby encapsulating the nucleic acid within the VLP. In other embodiments, the ratio of HPV-capsid protein to reaction volume ranges from 0.2 μg HPV-capsid protein per 1 μl reaction volume to 0.6 μg HPV-capsid protein per 1 μl reaction volume. In yet other embodiments, the ratio of nucleic acid to HPV-capsid protein ranges from 0.5 μg nucleic acid per 1 μg HPV-capsid protein to 3.5 μg nucleic acid per 1 μg HPV-capsid protein. In yet other embodiments, the ratio of nucleic acid to reaction volume ranges from 0.2 μg nucleic acid per 1 μl reaction volume to 3 μg nucleic acid per 1 μl reaction volume.

The step of dissociating the VLP or capsid protein oligomers can be carried out in a solution comprising ethylene glycol tetraacetic acid (EGTA) and dithiothreitol (DTT),

wherein the concentration of EGTA ranges from 0.3 mM to 30 mM and the concentration of DTT ranges from 2 mM to 200 mM. In certain embodiments, the concentration of EGTA ranges from 1 mM to 5 mM. In certain embodiments, the concentration of DTT ranges from 5 mM to 50 mM.

The step of reassembling of capsid proteins into a VLP can be carried out in a solution comprising dimethyl sulfoxide (DMSO), CaCl₂ and ZnCl₂, wherein the concentration of DMSO ranges from 0.03% to 3% volume/volume, the concentration of CaCl₂ ranges from 0.2 mM to 20 mM, and the concentration of ZnCl₂ ranges from 0.5 μM to 50 μM. In certain embodiments, the concentration of DMSO ranges from 0.1% to 1% volume/volume. In certain embodiments, the concentration of ZnCl₂ ranges from 1 μM to 20 μM. In certain embodiments, the concentration of CaCl₂ ranges from 1 mM to 10 mM.

In certain embodiments, the loading method is further modified to stabilize the VLP, in that the loading reaction is dialyzed against hypertonic NaCl solution (e.g., using a NaCl concentration of about 500 mM) instead of phosphate-buffered saline (PBS), as was previously described. Surprisingly, this reduces the tendency of the loaded VLP to form larger agglomerates and precipitate. In certain embodiments, the concentration of NaCl ranges between 5 mM and 5 M. In certain embodiments, the concentration of NaCl ranges between 20 mM and 1 M.

In some embodiments, VLPs can be loaded with one or more therapeutic agents according to the methods described herein. In certain embodiments the therapeutic agent is a nucleic acid molecule capable of inducing RNA interference or a nucleic acid (e.g., plasmid or other vector) that is capable of expressing a nucleic acid molecule capable of inducing RNA interference. In some embodiments. VLP can be loaded with combinations of two or more therapeutic or diagnostic agents.

In some embodiments, the therapeutic agent is an inducer of RNA interference or other inducer of gene silencing. An inducer of RNA interference may be a siRNA, a shRNA, a hybrid nucleic acid molecule comprising a first part that comprises a duplex ribonucleic acid (RNA) molecule and a second part that comprises a single stranded deoxyribonucleic acid (DNA) molecule, a longer double-stranded RNA or a DNA construct for expression of siRNA or longer RNA sequences. Other inducers of gene silencing include inducers of DNA methylation, or ribozymes, or aptamers. In other embodiments, the therapeutic agent can be a modulator of gene expression such as a PNA (Peptide Nucleic Acid).

RNA interference (RNAi) is a process whereby the introduction of double stranded RNA (dsRNA) into a cell inhibits gene expression post-transcriptionally, in a sequence dependent fashion. RNAi can be mediated by short (for example 19-25 nucleotides) dsRNAs or small interfering RNAs (siRNA). dsRNA is cleaved in the cell to create siRNAs that are incorporated into an RNA-induced silencing complex (RISC), guiding the complex to a homologous endogenous mRNA, cleaving the mRNA transcript, and resulting in the destruction of the mRNA.

To induce RNA interference in a cell, dsRNA may be introduced into the cell as an isolated nucleic acid fragment or via a transgene, plasmid, or virus. In certain embodiments, VLP are used to deliver dsRNA to the target cells.

In some embodiments a short hairpin RNA molecule (shRNA) is expressed in the cell. A shRNA comprises short inverted repeats separated by a small loop sequence. One inverted repeat is complimentary to the gene target. The shRNA is then processed into an siRNA which degrades the target gene mRNA. shRNAs can produced within a cell with a DNA construct encoding the shRNA sequence under control of a RNA polymerase III promoter, such as the human H1, U6 or 7SK promoter. Alternatively, the shRNA may be synthesized exogenously and introduced directly into the cell, for example through VLP delivery. In certain embodiments, the shRNA sequence is between 40 and 100 bases in length or between 40 and 70 bases in length. The stem of the hairpin are, for example, between 19 and 30 base pairs in length. The stem may contain G-U pairings to stabilize the hairpin structure.

siRNA sequences are selected on the basis of their homology to the target gene. Homology between two nucleotide sequences may be determined using a variety of programs including the BLAST program (Altschul et al. (1990) J. Mol. Biol. 215: 403-10), or BestFit (Genetics Computer Group, 575 Science Drive, Madison, Wis., USA, Wisconsin 53711). Sequence comparisons may be made using FASTA and FASTP (see Pearson & Lipman, 1988. Methods in Enzymology 183: 63-98). Tools for design and quality of siRNAs, shRNAs and/or miRNAs are known in the art. Web-based online software system for designing siRNA sequences and scrambled siRNA sequences are for example siDirect, siSearch, SEQ2SVM, Deqor, siRNA Wizard (InvivoGen). The specificity can be predicted using for example SpecificityServer, miRacle. Target sequences can be researched for example at HuSiDa (Human siRNA Database), and siRNAdb (a database of siRNA sequences). Sequence comparison may be made over the full length of the relevant sequence, or may more preferably be over a contiguous sequence of about or 10, 15, 20, 25 or 30 bases. In certain embodiments, the degree of homology between the siRNA and the target gene is at least 75%, at least 80%, at least 85%, at least 90%, at least 95%, at least 97%, or at least 99%, or 100%. The siRNA may be between 10 bp and 30 bp in length, or between 20 bp and 25 bp, or the siRNA is 20, 21 or 22 bp in length.

The occurrence of RNAi can be detected by transfecting cultured cells with the siRNA, followed by RT-PCR of the mRNA of interest. Where RNAi is induced by the siRNA, levels of the mRNA of interest will be reduced in transfected cells as compared to control cells. A reduction in protein production can be confirmed by Western blotting of cell lysates followed by probing with an antibody reactive to the protein of interest.

siRNA molecules may be synthesized using standard solid or solution phase synthesis techniques which are known in the art.

In some embodiments, the siRNA has an overhang at one or both ends of one or more deoxythymidine bases to increase the stability of the siRNA within cells by reducing its susceptibility to degradation by nucleases.

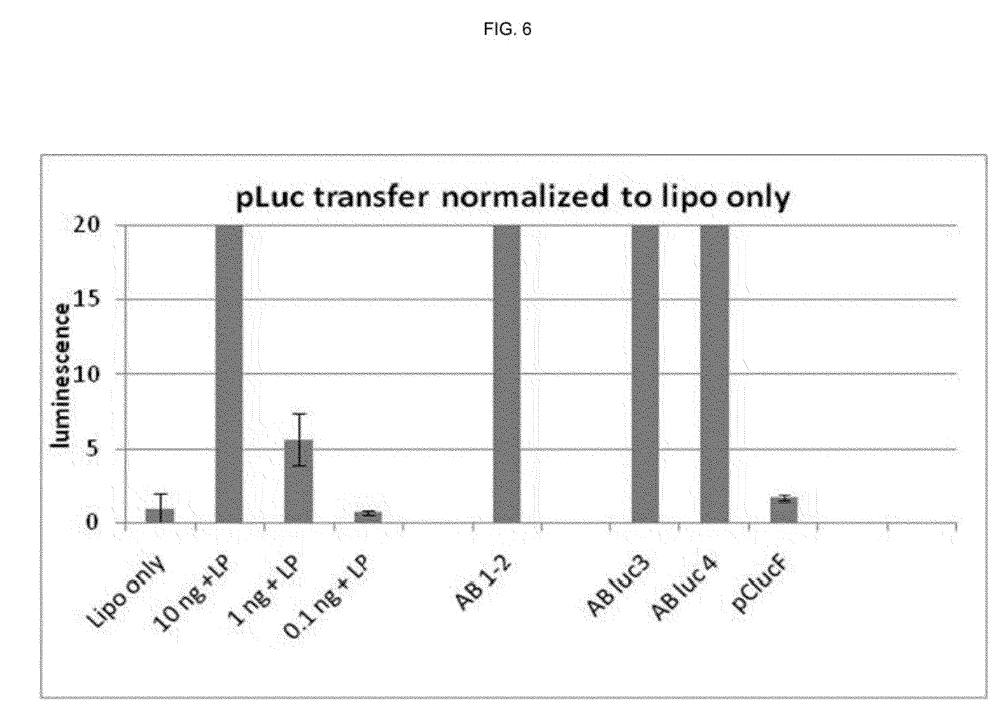

In some embodiments, the siRNA is a hybrid nucleic acid molecule comprising a first part that comprises a duplex ribonucleic acid (RNA) molecule and a second part that comprises a single stranded deoxyribonucleic acid (DNA) molecule. Preferred targets for the RNA interference would include disease causing genes e.g. oncogenes, inflammatory genes, regulatory genes, metabolic genes, viral genes. In one embodiment of the invention the target genes would be E6 & E7 HPV viral oncogenes (e.g., E7 siRNA, FIGS. 6A and 613). In other embodiments of the invention the target genes would be p53, Sirt-1, survivin, EGFR, VEGFR, VEGF, CTNNB 1 or other oncogenes.

Linkages between nucleotides may be phosphodiester bonds or alternatives, for example, linking groups of the formula P(O)S, (thioate); P(S)S, (dithioate); P(O)NR′2; P (O)R′; P(O)OR6; CO; or CONR′2 wherein R is H (or a salt) or alkyl (1-12C) and R6 is alkyl (1-9C) is joined to adjacent nucleotides through-O— or —S—.

Modified nucleotide bases can be used in addition to the naturally occurring bases. For example, modified bases may increase the stability of the siRNA molecule, thereby reducing the amount required for silencing. The term modified nucleotide base encompasses nucleotides with a covalently modified base and/or sugar. For example, modified nucleotides include nucleotides having sugars which are covalently attached to low molecular weight organic groups other than a hydroxyl group at the 3′position and other than a phosphate group at the 5′position. Thus modified nucleotides may also include 2′substituted sugars such as 2′-O-methyl-; 2-O-alkyl; 2-O-allyl; 2′-S-allyl; 2′-fluoro-; 2′-halo or 2; azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include alkylated purines and pyrimidines, acylated purines and pyrimidines, and other heterocycles. These classes of pyrimidines and purines are known in the art and include pseudoisocytosine, N4, N4-ethanocytosine, 8-hydroxy-N-6-methyladenine, 4-acetylcytosine, 5-(carboxyhydroxylmethyl) uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2-methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5-methoxyuracil, 2 methylthio-N6-isopentenyladenine, uracil-5-oxyacetic acid methyl ester; psueouracil, 2-thiocytosine, 5-methyl-2 thiouracil, 2-thiouracil, 4-thiouracil, 5-methyluracil, N-uracil-5-oxyacetic acid methylester, uracil 5-oxyacetic acid, queosine, 2-thiocytosine, 5-propyluracil, 5-propylcytosine, 5-ethyluracil, 5-ethylcytosine, 5-butyluracil, 5-pentyluracil, 5-pentylcytosine, and 2,6, diaminopurine, methylpsuedouracil, 1-methylguanine, 1-methylcytosine.

In some embodiments, siRNA molecules or longer dsRNA molecules may be made recombinantly by transcription of a nucleic acid sequence, for example contained within a vector as described herein. The vector may be any RNA or DNA vector.

The vector can be an expression vector, wherein the nucleotide sequence is operably linked to a promoter compatible with the cell. Promoters suitable for use in various vertebrate systems are well known in the art. For example, suitable promoters include viral promoters such as mammalian retrovirus or DNA virus promoters, e.g., MLV, CMV, RSV, SV40 IEP (immediate early promoter) and adenovirus promoters and metallothionein promoter. Strong mammalian promoters may also be used. It will be appreciated that variants of such promoters retaining substantially similar transcriptional activities may also be used.

In some embodiments, the vector may have at least two promoters, one to direct expression of the sense strand and one to direct expression of the antisense strand of the dsRNA. In other embodiments, two vectors may be used, one for the sense strand and one for the antisense strand. Alternatively the vector may encode RNAs which form stem-loop structures which are subsequently cleaved by the cell to produce dsRNA.

The nucleic acid construct may contain a specific cellular, viral or other promoter or repressor of gene expression. The promoter or repressor may be designed to reflect the context of the cell into which the construct is introduced. For example, the construct may contain a viral promoter so expression from the construct is dependent upon the presence of a viral protein, so that the construct is expressed only in viral-infected cells. Similarly, the construct may have a promoter or repressor specific to certain cell types or to certain developmental stages. For example, where the vector is for use in virally infected cell such as cells infected with HPV, a viral promoter which matches the disease-causing virus should be used, e.g., a HPV promoter (such as the promoter causing expression of HPV E6/E7) for HPV-infected cells. In such embodiments, the vector will only be expressed in the virally-infected cells.

Nucleic acids are highly charged and do not cross cell membranes by free diffusion. The hydrophilic character and anionic backbone of nucleic acids such as, for example, siRNAs reduces their uptake by the cells. In certain embodiments, nucleic acids (e.g. siRNA) may be loaded into VLP (e.g. HPV-VLP) to efficiently deliver them to a subject through administration of VLP. In certain embodiments, encapsulating the nucleic acid into a VLP increases cellular uptake, allows traversal of biological membrane barriers in vivo, and/or increases the bioavailability of the nucleic acid (e.g. siRNA).

In some embodiments, the agent loaded into the VLP is an anti-viral agent. In certain embodiments, the agent is an anticancer agent. In a preferred embodiment, the anticancer agent is a taxane.

In certain embodiments, the therapeutic agent that may be loaded into a VLP using the methods described herein is a chemotherapeutic agent, for instance, methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MM1270, BAY 12-9566, RAS farnesyl transferase inhibitor, farnesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZD0101, 1S1641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron/strontium derivative, Temodal/Temozolomide, Evacet/liposomal doxorubicin, Yewtaxan/Paclitaxel, Taxol/Paclitaxel, Xeload/Capecitabine, Furtulon/Doxitluridine, Cyclopax/oral paclitaxel, Oral Taxoid, SPU-077/Cisplatin, FIMR 1275/Flavopiridol, CP-358 (774)/EGFR, CP-609 (754)/RAS oncogene inhibitor, ISMS-182751/oral platinum, UFT (Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, Iodine seeds, CDK4 and CDK2 inhibitors, PARP inhibitors, D4809/Dexifosamide, Ifes/Mesnex/Ifosamide, Vumonn/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCl, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26) or Vindesine sulfate, but it is not so limited.

In certain embodiments, the therapeutic agent that may be loaded into a VLP using the methods described herein is an immunotherapeutic agent, for instance, Ributaxin, Herceptin, Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GN1-250, EMD-72000, LymphoCide, CMA 676, Monopharm-C, 4B5, ior egf.r3, ior c5, BABS, anti-FLK-2, MDX-260, ANA Ab, SMART 1D10 Ab, SMART ABL 364 Ab or ImmuRAIT-CEA, but it is not so limited.

In certain embodiments, the therapeutic agent that may be loaded into a VLP using the methods described herein is an antiviral agent. Examples of anti-viral agents are: Polysulfates (PVAS), Polysulfonates (PVS), Polycarboxylates, Polyoxometalates, Chicoric acid, zintevir, cosalane derivatives, Bicyclams (i.e., AMD3100), T-22, T-134, ALX-40-4C, CGP-64222, TAK-779, AZT (azidothymidine), ddI, ddC, d4T (didehydrodideoxythymidine), 3TC (3′-thiadideoxycytidine), ABC, and other ddN (2′,3′-dideoxynucleoside) analogs, Nevirapine, delavirdine, efavirenz, emivirine (MKC-442), capravirine, thiocarboxanilide UC-781, acyclovir, valaciclovir, penciclovir, famciclovir, bromovinyldeoxyuridine (BVDU, brivudin), Cidofovir, Adefovir dipivoxil, Tenofovir disoproxil, Ribavirin, valacyclovir, gancyclovir, formivirsen, foscarnet, EICAR (5-ethynyl-1-β-D-ribofuranosylimidazole-4-carboxamide), Mycophenolic acid, Neplanocin A, 3-deazaneplanocin A, 6′-C-methylneplanocin A, DHCeA (9-(trans-2′,trans-3′-dihydroxycyclopent-4′-enyl)adenine), or c³DHCeA (9-(trans-2′,trans-3′-dihydroxycyclopent-4′-enyl)-3-deazaadenine), as described, for example, in De Clercq J Pharmacol Exp Ther 2001, 297: 1-10, incorporated by reference herein, but it is not so limited.

Aspects of the invention are not limited in its application to the details of construction and the arrangement of components set forth in the preceding description or illustrated in the examples or in the drawings. Aspects of the invention are capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EXAMPLES

### Example 1

### Production and Purification of Capsid Proteins in Host Cells and In Vitro Reassembly into VLPs

Suspension cultures of Sf9 insect cells were maintained in serum-free Sf-900™ II medium (Invitrogen, Lide Technologies) and expanded from shake flasks to WAVE bioreactorsml (GE Healthcare Lifesciences). Approximately 2 L of shake flask culture was utilized to seed the 10 L WAVE Bioreactors™ at an initial density of 4×10⁵ cells/ml.

Once the actively growing culture reached a density between 1.5-2×10⁶ cells ml, it was infected with a recombinant baculovirus stock for HPV16L1 or HPV16/31 mutant and a HPV16L2 at an MOI of 5. Recombinant baculovirus stocks were produced, as described herein (Table 1)

According the present invention, an overview of an exemplary protocol for generating Baculovirus generation and preparing a high-titer stock preparation is described as follows. Transform DH10Bac Competent Cells with pFastBac construct and heat shock the mixture. Serial dilute the cells using SOC medium to 1:10, 1:100 and 1:1000 dilutions. Grow cultures for 4 hours at 37 C at 250 rpm. Streak the 1:10, 1:100 and 1:1000 dilutions onto selective plates of LB-Agar/Kan/Tet/Gent/X-gal/PTG. Incubate plates for 48 hours at 37 C. Select three white colonies. Grow each culture O/N at 37 C at 250 rpm in LB plus Kan, Gent. & Tet. Harvest cell pellets by centrifugation and isolate recombinant Bacmid by alkaline lysis method. Determine Bacmid concentration by 260:280. Tranfect Sf9 cells with Bacmid/cellfectin complex and plate. Incubate plates for four days in a humidified 27 C tissue culture incubator. Transfer conditioned media to 30 ml SF Sf9) culture. Grow culture 3-5 days. Monitor for cell viability and cell diameter using Vi-Cell. Harvest conditioned media and cell pellet when viability is less than 75%. Perform titer (BacPAK RapidTiter Kit) and Western Plot analysis. Expand recombinant virus by infecting a 1 L culture Sf9 cells at an MOI of 0.1 with the best expressing Baculovirus clone. Harvest conditioned media by centrifugation once viability has dropped less than 75%. Perform titer analysis using RapidTiter Kit.

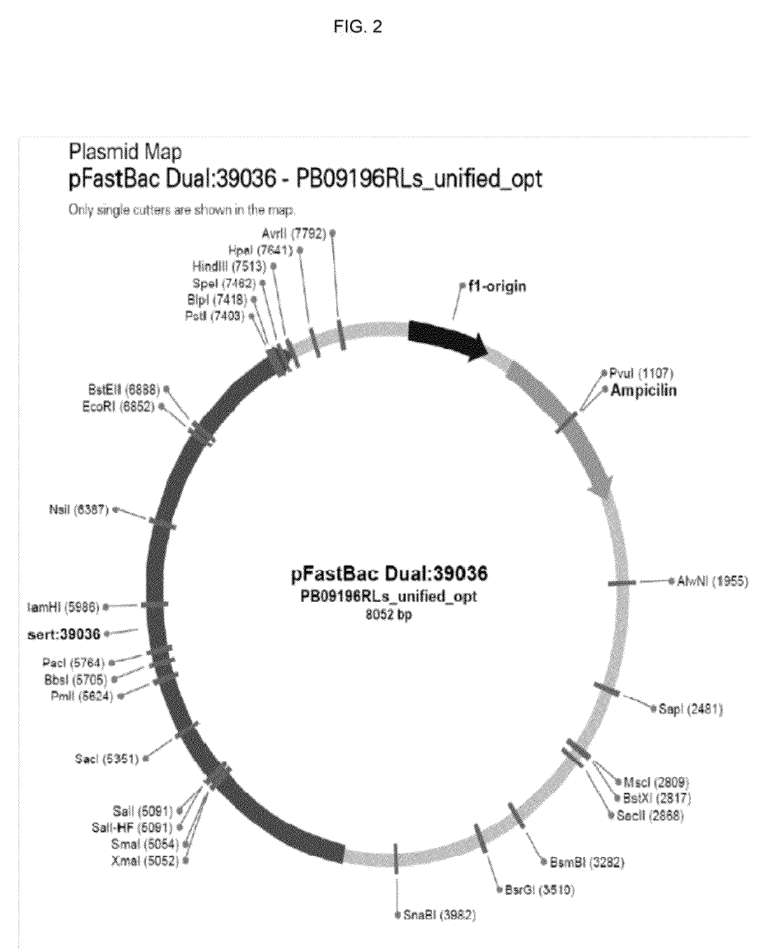

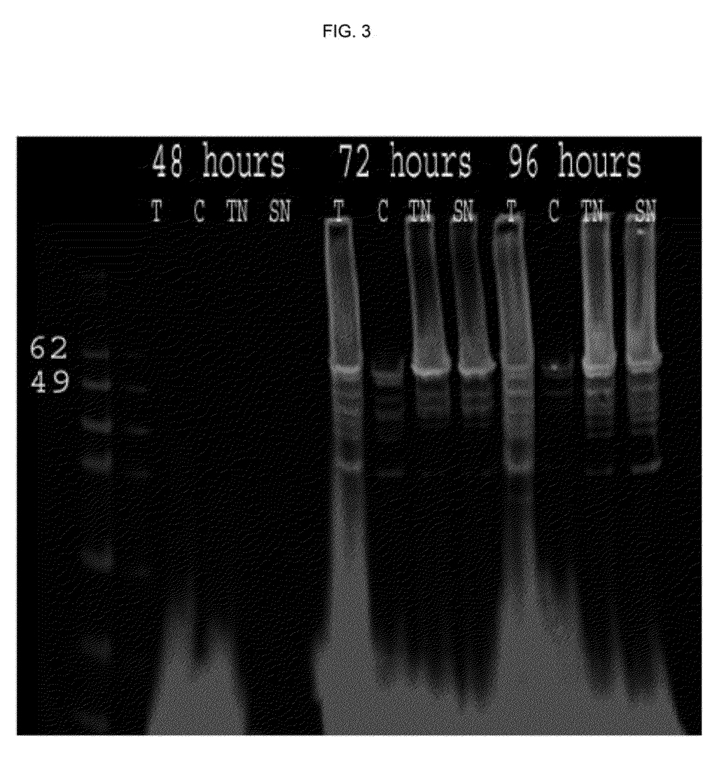

To generate the recombinant baculovirus for HPV16/31 L1 production, the pFastBac™ plasmid (Invitrogen, Life Technologies) (FIG. 2) containing 16/31 L1 DNA sequence (SEQ ID NO: 1) was used. To generate the recombinant baculovirus for HPV16L2 production, the pFastBac™ plasmid containing L2 DNA sequence (SEQ ID NO: 2) was used. During recombinant protein production, the bioreactor was monitored daily for cell count viability, cell size and pH. Seventy-two hours post-infection, the cell pellet was obtained by tangential-flow filtration, washed in PBS, re-pelleted by centrifugation, and stored at −80° C. Western blot using protein-specific antibodies for L1 and L2 proteins were then used to verify the presence of the recombinant protein.

Following verification of expression, purification of HPV capsomeres produced above was performed. Cells were thawed on ice and then resuspended in ice-cold lysis buffer (PBS plus 0.5% Nonidet™ P-40 (Shell Chemical Co.)) at a ratio of 10 ml of buffer per gram of cell

TABLE 1
Transform DH10Bac Cells with pFastbac Construct
Use pFastbac Dual construct generated at DNA2.0 to transform DH10Bac cells by heat shock
method (i.e. 1 ng, pFactbac construct in 100 ul of cells. Incubate for 30 minutes on ice. Heat
at 42 C. for 45 seconds. Chill on ice for two minutes). Grow cultures at 37 C., 225 rpm in SOC
media for four hours. Prepare 1:10, 1:100, and 1:1000 dilutions of culture. Plate dilutions on
Bac-to-Bac selective plates. Incubate plates at 37 C. for two days.
Purify Recombinant Bacmid
Select three well defined white colonies from the Bac-to-Bac selective plates and culture the
cells in selective LB media overnight. Collect bacterial cells by centrifugation (14Kxg. 3
minutes). Resuspend cell pellets in P1 buffer. Lyse cells by the addition of an equal volume
of P2 buffer. Incubate at room temperature for five minutes. Precipitate genomic DNA and
protein by addition of a half colume of P3 bugger and incubation on ice for five minutes.
Remove precipitated contaminants by centrifugation (14K x g; 10 minutes) and reserve
supernatant. Precipitate the bacmid by addition of an equal volume of Isopropanol followed
by an overnight incubation at 20 C.. Pellet bacmid by centrifugation. Wash pelleted bacmid
with 70% ethanol. Let pellet air dry. Resuspend pellet in TE. Determine yield and purity by
OD260-OD280.
Transfect Sf9 Cells With Recombinant Bacmid
For each bacmid prepare a 6-well plate with 1 × 20e6 cells per well in standard growth media
(i.e. Sf-900 II). Allow cells to attach to the plate for at least 1 hour. In a BSC, prepare
bacmid Cellfectin complex by mixing 1 ug of bacmid that has been diluted with 100 ul of
Grace's media with 6 ul of cellfectin transfection reagent that has been diluted with 100 ul of
Grace's media. Let complexes form for 30 minutes at room temperature. Remove media
from the cells in upper left corner well, dilute bacmid cellfectin complex with 800 ul of
Grace's media, add transfection solution to the upper left corner well. Place plates into a
humidified incubator at 27 C.. After five hours, remove transfection solution from the cells in
the upper left corner well and add 2 ml of growth media (i.e. Sf-900 II). Return plates to the
humidified incubator. Check cells daily under a microscope to confirm transfection (cells
should not grow as fast as control cells and should increase in diameter, and eventually the
cells should show signs of lysing). After four days, harvest P0 viral stock (i.e. conditioned
media from upper left corner well).
Amplify P0 Baculoviral Stock:
For each baculoviral stock, add 1 ml of the P0 viral stock to a 30 ml culture in a 125 ml shake
flask of Sf9 cell at a cell density of 1e6 cells/ml. An additional SF is utilized as a negative
control and 1 ml of growth media added. Shaking incubator parameters are 120 rpm and
27.5 C.. Cultures are monitored daily with the Vi-Cell for cell density, cell viability, and
diameter. In a proper infection, within 48 hours the insect cell culture should have
significantly lower cell density and cell viability and increased cell diameter. Cultures are
maintained for three to five days and harvested by centrifugation (2500 x g, 10 minutes) once
viability has dropped below 75%. Transfer the conditioned media (P1) viral stock to a fresh
tube and store at 4 C.. Reserve cell pellet for Western analysis. Determine titer for the p1
viral stock using the Clontech BacPAK Rapid Titer Ket according to manufacturer's
protocol.
Expand P1 Baculoviral Stock
For the best expressing baculoviral stock (i.e. Western Analysis), add 1.5e8 pfu of P1 viral
stock to a 1 L culture of Sf9 cells in a 3 L Shake Flask at 1.5e6 cells per ml (i.e. MOI of 0.1).
Shaking incubator parameters are 120 rpm and 27.5 C.. Cultures are monitored daily with Vi-
Cell for cell density, cell viability, and cell diameter. Cultures are maintained for two to five
days and harvested by centrifugation (2500 x g, 10 minutes) once viability has dropped below
75%. Transfer the conditioned media (P1) viral stock to a fresh sterile bottle and store at 4 C..
Determine titer for the P2 viral stock using the Clontech BacPAK Rapid Titer Kit according
to manufacturer's protocol.

paste. Resuspended cells were then incubated on ice for 15 min. After chemical lysis, nuclei were isolated by centrifugation (3000×g for 15 min) and then resuspended in ice-cold PBS without detergent. Capsid proteins were then solubilized from the isolated nuclei with three 15 s bursts of a sonicator at 50% maximal power. Insoluble material was then clarified by centrifugation (1000×g for 10 min) and the resulting supernatant was diafiltered into TMAE buffer by TFF using a 100 kDa molecular weight cut-off titter. Western Blot was used to demonstrate that the majority of the capsid proteins was localized in the nuclear fraction. (FIG. 6)

Capsid proteins were then loaded onto a TMAE column, washed, and eluted using a linear salt gradient. Early fractions containing the proteins of interest were then pooled, dialyzed into disassociation buffer, and concentrated to a final concentration of 1 mg/ml.

Purified capsid proteins were then assembled in a cell free system together with a plasmid (pENTR™/U6 plasmid (Invitrogen, Life Technologies)) expressing an shRNA construct containing the short hairpin RNA sequence generated using primer sequences (SEQ ID NO: 3 and SEQ ID NO: 4) to create VLP encapsulating the shRNA using the following loading protocol.

Loading Protocol

In a clean 15 ml conical tube the following reagents were added and incubated at 37° C. for 30 min: 200 μg of capsomere protein; 100 μg pENTR™/U6/shRNA plasmid; 0.5 μl DMSO; and 15 μl Solution 2 (150 mM Tris-HCl pH 7.5, 450 mM NaCl, 330 μl dH₂O), brought up to a total volume of 150 μl.

Solution 3 (2 mM CaCl₂, 5 μM CaCl₂, 50 mM Tris-HCl pH 7.5, 150 mM NaCl, 434 μL dH₂O) was then added to the above mixture and incubated at 37° C. for 30 min.

Solution 4 (4 mM CaCl₂, 10 μM CaCl₂, 50 mM Tris-HCl pH 7.5, 150 mM NaCl, 1224 μl dH₂O) was then added to the above mixture and incubated at 37° C. for 2 hrs.

The mixture was then dialyzed in 1×PBS at 4° C. overnight.

### Example 2

### Production of Mutant L1* and L2 Capsid Proteins in Mammalian Cell System

Similarly to Example 1 described above, a mammalian culture system is used to produce mutant L1*(16/31) and L2 capsid proteins. Plasmids containing human-optimized codon sequences are used for this purpose (SEQ ID NO: 5), and a general protocol is followed (Buck, C. B., et al. (2005) Methods Mol. Med., 119: 445-462, which reference is incorporated herein).

### Example 3

### Assembly into VLPs from Capsid Proteins

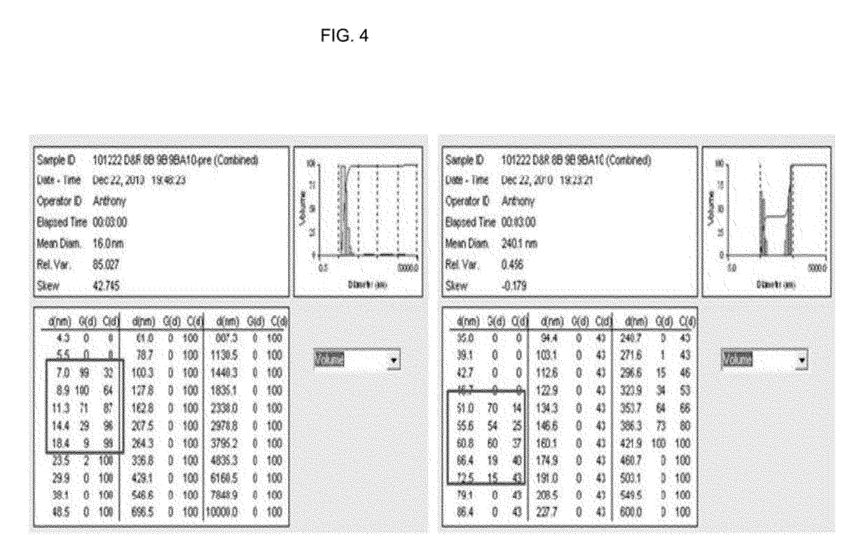

Capsid proteins isolated from insect cells were assembled into VLPs as described. Dynamic light scattering (DLS) demonstrates presence of capsid proteins in monomeric and oligomeric forms (<10 nm) after harvest and prior to the loading procedure. After the reassembly in presence of the nucleic acid payload, VLPs are seen by DLS (50-70 nm diameter) (FIG. 4).

### Example 4

### Functional Transfer of Luciferase Expression

Results show functional transfer of luciferase expression. VLPs were generated using different production methods to compare efficacy. Transfection of luciferase plasmid (pClucF) using standard lipofectamine transfection at various plasmid amounts (0.1 ng/well, 1 ng/well, 10 ng/well) was used to create a range of positive controls. 10 ng of pClucF plasmid was used without transfection reagent as a reagent/background control.

AB1-2 refers to HPV16L1L2 VLP generated using the methods described above, where a single plasmid like p 6sheLL (SEQ ID NO: 6) was used to co-express wildtype HPV L1 and L2 proteins.

Capsid proteins were purified, as described above, from 293 cells transfected with the co-expression plasmid for L1 and L2. Capsid proteins were then subjected to the following loading protocol, thereby forming loaded

Loading Protocol

In a clean 15 ml conical tube the following reagents were added and incubated at 37° C. for 30 min: 200 μg of capsid proteins, 100 μg pClucF, 0.5 μl DMSO, 15 μl Solution 2 (150 mM Tris-HCl pH7.5, 450 mM NaCl, 330 μl dH₂O), brought up to a total volume of 150 μl.

Solution 3 (2 mM CaCl₂, 5 μM CaCl₂, 50 mM Tris-HCl pH 7.5, 150 mM NaCl, 434 μL dH₂O) was then added to the above mixture and incubated at 37° C. for 30 min.

Solution 4 (4 mM CaCl₂, 10 μM CaCl₂, 50 mM Tris-HCl pH 7.5, 150 mM NaCl, 1224 μl dH₂O) was then added to the above mixture and incubated at 37° C. for 2 hrs.

The mixture was then dialyzed in 1×PBS at 4° C. overnight.

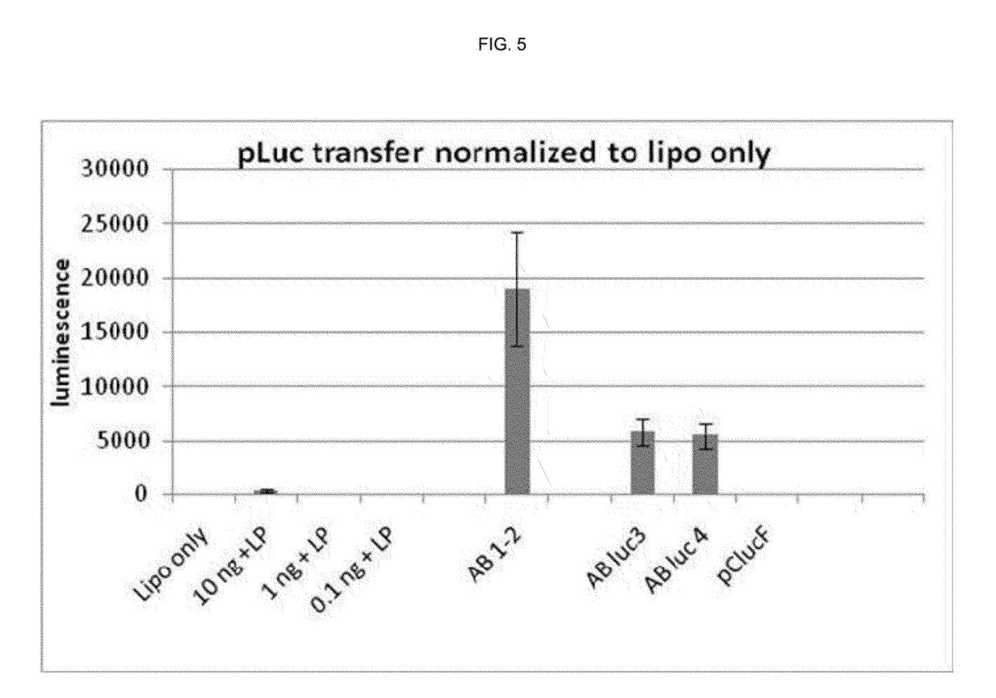

Loaded VLP were then used to treat Hela cells in 96 well plates and luciferase signal was read after 48 hrs (Table 2, FIGS. 5 and 6).

AB luc3 and AB luc4 were produced in 293 cells after transfection with the p16sheLL plasmid as pseudovirions (PSV) already encapsulating the payload plasmid (pClucF) (Buck, C. B., et al. (2005) Methods Mol. Med., 119: 445-462). Results showed superior transfer of plasmid when the reassembly loading method was used (AB 1-2) compared with VLPs that were loaded through packaging of plasmid in the host cells (A13 luc 3 and AB luc 4).

TABLE 2

| Sample | Average | STDEV |
|---|---|---|
| Lipo only | 1 | 1 |
| 10 ng + LP | 338.4552177 | 114.5688758 |
| 1 ng + LP | 5.61254622 | 1.747839908 |
| 0.1 ng + LP | 0.732641742 | 0.135130943 |
| AB 1-2 | 19011.91454 | 5216.078827 |
| AB luc3 | 5769.104355 | 1178.278814 |
| AB luc 4 | 5487.777321 | 1115.096887 |
| pClucF | 1.639379622 | 0.218550273 |

TABLE 3
Materials

| Item | Manufacturer | Catalog |
|---|---|---|
| pFastbac Dual: 39036 | DNA 2.0 | 39036 |
| (PB09196RLs_unified_opt) | | |
| Bac-to-Bac Dual vector | Invitrogen | 10712024 |
| MAX Efficiency Chemically | Invitrogen | 10361-012 |
| Competent DH10Bac | | |
| LB Broth | Amresco | J106 |
| Agar | Amresco | J637 |
| Kanamycin Sulfate | Calbiochem | 420311 |
| Gentamicin | Gibco | 15710 |
| Tetracycline Hydrochloride | Sigma | T7660 |
| Bluo-gal | Invitrogen | 15519-028 |
| Isopropylthis-B-galactoside | Inalco | 1758-1400 |
| (IPTG) | | |
| RNase A | | |
| P1 Buffer | Qiagen | 1014858 |
| P2 Buffer | Qiagen | 1014950 |
| P3 Buffer | Qiagen | 1014965 |
| Isopropanol | Malinkrodt | 3032-22 |
| Ethanol | Signma | E7023 |
| TE Buffer | Qiagen | 1018456 |
| Cellfectin reagent | Invitrogen | 10362-010 |
| Sf9 Cells | Gibco | 11496-015 |
| Sf-900 II SFM | Gibco | 10902-096 |
| Grace's Insect Cell Culture | Gibco | 11595-030 |
| Medium | | |
| BacPak Rapid Titer Kit | Clontech | 631406 |
| Mouse anti-6XHis antibody | Clontech | 631212 |
| Qdot 800 goat anti-mouse IgG | Invitrogen | Q1107MP |
| conjugate | | |
| Acetone | J.T.Baker | 9002-03 |
| Formaldehyde | VWR | VW3408-1 |
| Dimethylformamide | Sigma-Aldrich | 319937 |

TABLE 4
Equipment

| Item | Manufacturer/Model | Equipment # |
|---|---|---|
| Microbial Biosafety Cabinet | Forma Scientific/1184 | PB0138 |
| Shaking Microbial Incubator | NBS/PsycroTherm | PB0045 |
| Microcentrifuge | Eppendorf/5415D | PB0159 |
| UV/Vis Spectrophotometer | Agilent 8453 | PB0090 |
| Insect Biosafety Cabinet | Baker Co./SterilGARD III | 5007-0000 |
| Humidified Incubator | Forma Scientific/3326 | PB0013 |
| Microscope | Olympus/1X70 | PB0075 |
| Shaking Insect Incubator | NBS/Innova 4000 | PB0044 |
| Cell Analyzer | Beckman Coulter/Vi-Cell | PB0085 |
| | XR | |
| Table Top Centrifuge | Beckman/Allegra X-15R | PB0160 |
| Western Imaging Station | Li-Cor/Odyssey | PB0073 |

### Example 5

### Topical Therapeutic Treatment or Alopecia with BetaHPV Pseudovirions

In accordance with a preferred embodiment of the present invention, a preferred method for treating alopecia preferably includes using a combination of betapapillomavirus viral shells (L1/L2) to deliver a DHT inhibitor as a therapeutic agent. In accordance with the present invention, the method preferably includes the steps of constructing a recombinant DNA molecule which contains a sequence encoding a papillomavirus L1 protein or a papillomavirus L2 protein or a combination of L1 and L2 proteins and transfecting a host cell with the recombinant DNA molecule. Preferably, the virus like particles may express papillomavirus L1 protein or L2 protein or a combination of L1 and L2 proteins in the host cell. Thereafter, the papillomavirus virus-like particles obtained from the transfected host cell may be purified which will cause the disassembling the L1 and L2 capsid proteins of the virus-like particles into smaller units. Preferably, it is these smaller disassembled L1 and L2 capsid proteins which may be loaded with a DHT inhibitor. Next, the loaded proteins may be reassembled to form a loaded virus-like particles comprising HPV protein with the DHT inhibitor and administered to the skin of an animal or a human subject. The method can be applied to enhance topical delivery of Minoxidil, Finasteride or other drugs to treat AGA topically through the skin and through the hair follicle.

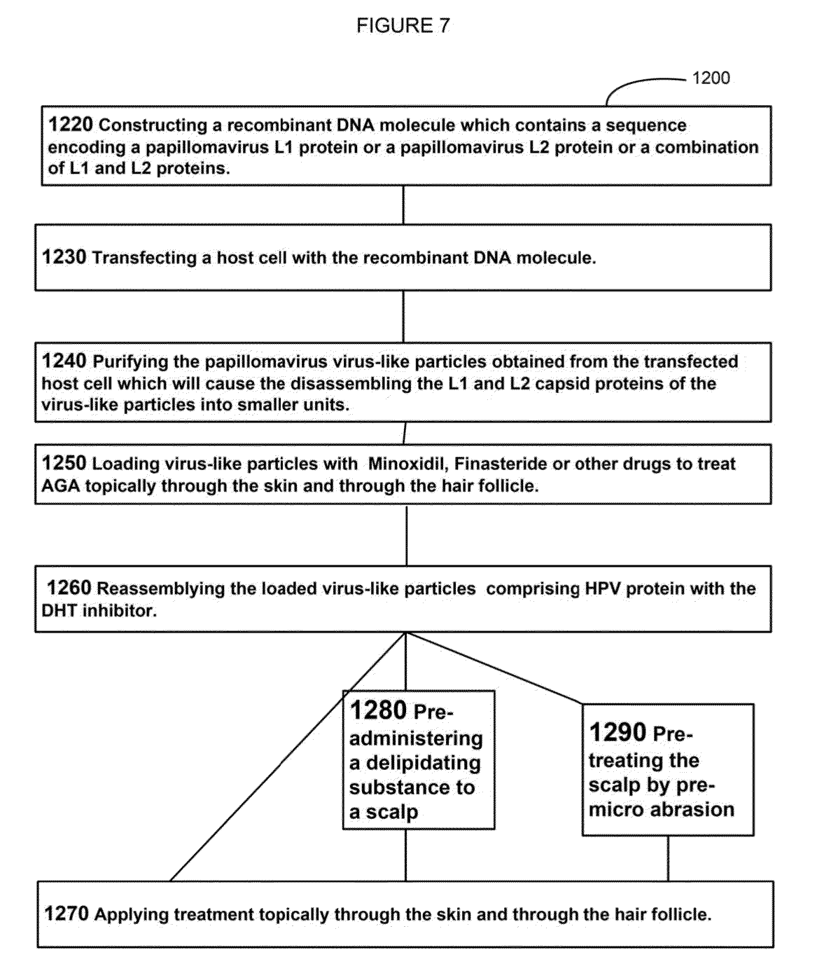

With reference now to FIG. 7, a method in accordance with an embodiment of the present invention, a preferred method of treating alopecia, will now be discussed. As shown in FIG., the present invention provides a method for treating alopecia 1200, which includes a first step in which a recombinant DNA molecule is constructed which contains a sequence for encoding a papillomavirus L1 protein or a papillomavirus L2 protein, or a combination of papillomavirus L1 and L2 proteins 1220. Thereafter, a host cell will be transfected with the recombinant DNA molecule 1230. Afterwhich, the transfected host cell will be treated to purify the papillomavirus virus-like particles causing the L1 and L2 capsid proteins to disassemble into smaller units 1240. At which time, an appropriate therapeutic agent or drug for treating AGA will be introduced into the proximity of the virus-like particle where the agent or drug for treatment will be loaded into the virus-like particles 1250. Thereafter, the loaded virus-like particles comprising HPV protein with an effective DHT inhibitor for treating alopecia may be reassembled 1260. Finally, the treatment may preferably be topically applied through the skin and the hair follicles 1270 for the treatment of alopecia.

As further shown in FIG. 7, in accordance with another preferred embodiment of the present invention, the above method further includes a step to pre-treat the area of the skin. In step 1280, the area of the scalp to be treated for alopecia may preferably be prepared by pre-administering a delipidating substance to the region of the scalp before applying the treatment of HPV pseudo-virus particles loaded with a DHT inhibitor to the scalp of a human subject.

As further shown in FIG. 7, in accordance with another preferred embodiment of the present invention, the above method also further includes another pre-treatment of the scalp. In step 1290, the area of the scalp to be treated for alopecia may be pre-treated by micro-abrasion before applying the treatment of HPV pseudo-virus particles loaded with a DHT inhibitor to the scalp of a human subject

The current invention can also be applied for hair removal. In one embodiment of the invention, a siRNA (short interfering nucleic acid) molecule may be formulated as a composition as described in U.S. Patent Application Publication No. 2007/0179104 A1 (which is hereby incorporated by reference herein). In this embodiment, the formulated composition which may be loaded into the betaHPV pseudovirions and transdermally administered may include such siRNA formulations which are generally referred to as “lipid nucleic acid particles” (LNP) which include membrane disruptive agents in which the siRNA molecule is complexed with a cationic lipid and helper lipid molecule; or formulated with polyethylenimine derivatives, including for example grated PEIs such as galactose PEI, cholesterol PEI, antibody derivatized PEI, and polyethylene glycol PEI (PEG-PEI) and/or derivatives thereof

While the above descriptions regarding the present invention contains much specificity, these should not be construed as limitations on the scope, but rather as examples. Many other variations are possible. Accordingly, the scope should be determined not by the embodiments illustrated, but by the appended claims and their legal equivalents. For example, alternative viral vectors may be used in place of the betapapillomavirus. For example, alternative viral vector may include herpes virus vectors.

The claims defining the invention are as follows:

1. A composition for transdermal drug delivery comprising a HPV protein and a therapeutic agent to treat a skin related disease.

2. The composition of claim 1, wherein the HPV protein is L1 or L2.

3. The composition of claim 1, wherein the HPV protein is L1 and L2.

4. The composition of claim 1, wherein the HPV is from the genus betapapillomavirus.

5. The composition of claim 1, wherein the HPV protein is HPV5.

6. A composition for transdermal drug delivery for the treatment of alopecia consisting essentially of an HPV protein and a DHT inhibitor as a therapeutic agent.

7. The composition of claim 6, wherein the DHT inhibitor is a small molecule.

8. The composition of claim 6, wherein the DHT inhibitor is RNAi.

9. A method for treating alopecia using a combination of betapapillomavirus viral shells (L1/L2) to deliver a DHT inhibitor as a therapeutic agent, the method comprising essentially the steps of:
constructing a recombinant DNA molecule that contains a sequence encoding a papillomavirus L1 protein or a papillomavirus L2 protein or a combination of L1 and L2 proteins;
transfecting a host cell with the recombinant DNA molecule;
expressing papillomavirus L1 protein or L2 protein or a combination of L1 and L2 proteins in the host cell;
obtaining the papillomavirus virus-like particles from the transfected host cell;
purifying the virus-like particles;
disassembling the L1 and L2 capsid proteins of the virus-like particles into smaller units;
loading said disassembled L1 and L2 capsid proteins with a DHT inhibitor;
reassembling the loaded proteins to form a loaded virus-like Particles comprising HPV protein with the DHT inhibitor; and
administering said loaded virus-like particles to the skin of an animal or a human subject.

10. The method according to claim 9, wherein the method includes pre-treating the scalp with a delipidating substance before treating for alopecia.

11. The method according to claim 9, wherein the method includes treating the scalp with a pre-micro abrasion before treating for alopecia.
